(19) United States
(12) Patent Application Publication
(10) Pub. No.: US 2011/0201103 A1
(43) Pub. Date: Aug. 18, 2011

(54) System For Synergetic Expression Of Multiple Small Functional RNA Elements

(75) Inventors: Gangning Liang, Rowland Heights, CA (US); Peter A. Jones, La Canada, CA (US); Jeffrey M. Friedman, Los Angeles, CA (US)

(73) Assignee: UNIVERSITY OF SOUTHERN CALIFORNIA, Los Angeles, CA (US)

(21) Appl. No.: 13/057,470

(22) PCT Filed: Aug. 7, 2009
(86) PCT No.: PCT/US2009/053203

§ 371 Date: Mar. 30, 2011

Related U.S. Application Data
(60) Provisional application No. 61/087,128, filed on Aug. 7, 2008.
(60) Provisional application No. 61/100,646, filed on Sep. 26, 2008.

(51) Int. Cl. C12N 15/63

(52) U.S. Cl. 435/320.1

(57) ABSTRACT

The present invention relates in general to microRNAs (miRNAs). More specifically, the invention relates to expression vectors comprising multiple miRNA families and clusters capable of targeting multiple oncogenic pathways.

The present application claims the benefit of the filing date of U.S. Provisional Application No. 61/087,128 filed Aug. 7, 2008, the disclosure of which is incorporated herein by reference in its entirety.

### FIELD OF THE INVENTION

The present invention relates in general to microRNAs (miRNAs). More specifically, the invention relates to microRNAs as targets for multiple genes or pathways in disease.

### BACKGROUND OF THE INVENTION

MicroRNAs (miRNA) are ˜22 nucleotide non-coding RNA molecules that function as endogenous repressors of target genes. The number of reported human miRNAs is over 450, but there are more than 1,000 predicted miRNAs (1). In general, RNA polymerase II transcribes a miRNA gene into a primary miRNA (pri-miRNA) that can be many kilobases long. The RNase III endonuclease Drosha processes the pri-miRNA in the nucleus to yield one or more precursor miRNAs (pre-miRNA) ˜70 nucleotides in length that form a stem-loop secondary structure. The pre-miRNA is exported to the cytoplasm where it is cleaved by the RNase III enzyme Dicer to generate the mature miRNA sequence, which is the substrate for subsequent repressive events. Mature miRNAs function in stable complexes with proteins of the Argonaute family, the core of the RNA-induced silencing complex (RISC). In animals miRNAs generally bind with imperfect complementarity to the 3′UTR of the target mRNA via the RISC complex. The RISC-miRNA-mRNA interaction results in gene repression that occurs by multiple mechanisms including enhanced mRNA degradation and translational repression (2). A recent study also indicates that miRNAs can act as endogenous activators of target genes when cells revert to an arrested state (3). Due to the promiscuity of miRNA binding to target mRNAs, each miRNA may control numerous genes and each mRNA may be controlled by many miRNAs (4). Developmental timing, cell death, proliferation, hematopoiesis, insulin secretion, and the immune response are just a few examples of critical biological events that depend on faithful miRNA expression (5).

### MicroRNAs and Cancer

A direct link between miRNA function and pathogenesis is supported by studies that revealed differential expression of miRNAs in tumors when compared to normal tissues. Discovering miRNAs that are differentially expressed between normal and tumor tissues can identify miRNAs that have a pathogenic role in cancer. The activation of oncogenic transcription factors, such as MYC, represents an important mechanism for altering miRNA expression (6). Genetic and epigenetic lesions can also alter miRNA expression, since miRNA up-regulation or down-regulation has been associated with genomic amplification, chromosomal deletions, point mutations, and aberrant promoter methylation (7-10). Although most of the aberrant miRNA expression observed in tumors is a secondary consequence of malignant transformation, some miRNAs have a causative role in tumorigenesis and can act as tumor suppressors or oncogenes. A miRNA whose target is a tumor suppressor gene or an oncogene will likely play a key role in tumorigenesis. If an overexpressed miRNA targets a tumor suppressor gene then it would suppress its target and would be an oncogenic miRNA. If a miRNA that normally suppresses an oncogene were deleted or otherwise down-regulated then it would be a tumor suppressor miRNA. Many well-studied miRNAs have had their functional roles during tumorigenesis confirmed by in vitro and/or in vivo studies and are therefore considered strong candidate tumor suppressors and oncogenes.

### MicroRNAs as Tumor Suppressors

In cancer, the expression of most miRNAs is decreased. Some of these down-regulated miRNAs may be tumor suppressor genes. Tumor suppressor miRNAs usually suppress tumor development by inhibiting oncogenes and/or genes that control cell differentiation or cell death. The miRNA clusters or families considered to be tumor suppressors and therefore most relevant to this proposal are described below:

The miR-34 family The p53 pathway acts as a sensor for many cancer-related signals, such as DNA damaging agents, radiation, oxidative stress, and activation of oncogenes. These signals affect cell proliferation, cell death, DNA repair, and angiogenesis through the function of p53 as a sequence-specific transcriptional regulator. Recent studies provided by several groups have linked the miR-34 family (miR-34a, miR-34b, miR-34c) to p53 by profiling miRNAs from wild-type and p53-null mice (11), human lung cancer cell lines with a temperature-sensitive TP53 allele (12), genotoxic stress in a p53-dependent manner (13), and p53 ChIP on chip (14). In all of these studies, the miR-34 family was identified as a target of p53. The miR-34 family can mediate induction of apoptosis, cell cycle arrest, and senescence by p53. This is the first time an interaction between proteins and non-coding RNAs has been shown in this crucial tumor suppressor pathway (15). Deletions of members of the miR-34 family have been reported in human cancers. miR-34a is located within 1p36, a region frequently deleted in many cancer types including neuroblastoma (16-18). In humans, mutations in p53 are found in nearly all types of cancers (19), thus the selective pressure to lose the miR-34 family may be relieved by frequent mutations in p53.

The let-7 family Let-7 is highly conserved in animals and it was originally identified in C. elegans by a mutant screen for genes that regulate developmental timing (20). The loss of function of let-7 prevents the normal transition of late larval to adult cell fate in C. elegans. This evidence raised the possibility that these miRNAs may regulate cellular proliferation and differentiation in humans. Indeed, several studies have suggested that human let-7 has a role as a tumor suppressor. Inappropriate expression of let-7 results in oncogenic loss of differentiation. In humans, let-7 is located at a frequently deleted chromosomal region in various cancers (7). Expression levels of let-7 were frequently reduced in both in vitro and in vivo lung cancer studies (21). Let-7 represses the expression of oncogenic components, such as RAS, MYC, and HMGA2, by targeting their mRNA for translational repression and overexpression of let-7 in cancer cells can inhibit cancer cell growth (22, 23). A recent study also indicated that let-7 can regulate self renewal and tumorigenicity of breast cancer cells (24).

miR-15a and miR-16-1 The first evidence that aberrant miRNA expression was involved in human cancer occurred in chronic lymphocytic leukemia (CLL). The 13q14 locus is deleted in over half of CLLs and this coincided with down-regulation of miR-15a and miR-16-1 which are located in this region (25). The loss of function of miR-15a and 16-1 is not only common in CLL but also in other cancers including prostate cancer, lymphoma, and multiple myeloma (7, 25, 26). The tumor suppressor function of these miRNAs is mediated by their ability to down-regulate the anti-apoptotic protein BCL2. Loss of miR-15a and 16-1 correlates with BCL2 overexpression and overexpression of these miRNAs leads to down-regulation of the endogenous protein and induction of apoptosis in CLL cells (27). Furthermore, the 3′ UTR of the BCL2 transcript has potential binding sites for these miRNAs and reporter constructs containing the BCL2 3′ UTR are down-regulated after co-expression of miR-15a and 16-1.

miR-143 and miR-145 miR-143 and miR-145 reside in a genomic cluster similar to that encoding miR-15a and rniR-16-1 and are down-regulated in cancer including colon cancer and B-cell malignancies (28, 29). Moreover, the introduction of either precursor or mature miR-143 and miR-145 into cancer cells with low expression of miR-143 and miR-145 results in significant growth inhibition (28, 29). A recent study also indicates that miR-145 targets the insulin receptor substrate-1 gene (IRS-1) and inhibits cell growth in colon cancer cell lines (30).

The miR-29 family Both overexpression of DNA methyltransferases and aberrant DNA methylation are commonly associated with cancer and may play a variety of roles in carcinogenesis (31, 32). Hypermethylation is responsible for the silencing of tumor suppressor genes in many cancers and could be a target for epigenetic therapy (33). DNA methylation changes are controlled by DNA methyltransferases (DNMTs). There are three catalytically active DNMTs; DNMT1, DNMT3A, and DNMT3B. DNMT1 is a copying or maintenance enzyme whereas DNMT3A and DNMT3B are responsible for the de novo methylation of previously unmethylated DNA during development. High levels of expression of DNMT1, DNMT3A, and DNMT3B are reported in various cancers. Inhibitors of DNA methylation, such as 5-aza-2′-deoxycytidine (5-Aza-CdR), inactivate DNMTs and rapidly reactivate the expression of genes that have undergone epigenetic silencing, particularly if this silencing has occurred in a pathological situation. Fabbri et al. used lung cancer cell lines to discover that the miR-29 family (miR-29a, miR-29b, and miR-29c) translationally down-regulated DNMT3A and DNMT3B, induced re-expression of methylation-silenced tumor suppressor genes, and restored normal methylation patterns (34). Furthermore, the overexpression of miR-29a, miR-29b, or miR-29c can inhibit the tumorigenicity of lung cancer in vitro and in vivo.

The miR-127 cluster Studies have shown that miRNAs are transcribed by RNA Pol II and the structure of pri-miRNAs includes a 7-methylguanosine cap and a poly(A) tail which is the same as a regular protein coding gene (35). Moreover, expression of miRNAs occurs in a tissue and tumor specific manner just like epigenetic changes including DNA methylation and histone modifications. These findings led us and others to find that some miRNAs are regulated by epigenetic alterations such as DNA methylation and histone modifications (10, 36-40). In our study, expression profiling of a bladder cancer cell line revealed that 17 out of 313 human miRNAs were upregulated more than 3-fold by treatment with the DNMT inhibitor and chromatin-modifying drugs 5-Aza-CdR and 4-phenylbutyric acid (PBA), respectively. One of these, miR-127, is embedded in a CpG island and was highly induced from its own promoter after treatment. miR-127 is usually expressed as part of a 4 kb miRNA cluster (miR-431, miR-433, miR-127, miR-432, and miR-136) in normal cells but not in cancer cells, suggesting that it is subject to epigenetic silencing. In addition, the proto-oncogene BCL6, a potential target of miR-127, was translationally down-regulated after both drug treatment and overexpression of miR-127 in cancer cell lines. These studies suggest that DNA demethylation and histone deacetylase inhibition can activate expression of miRNAs that may act as tumor suppressor, such as miR-127.

### MiRNAs as Oncogenes

Some miRNAs that are overexpressed in tumors may be oncogenes. These oncogenic miRNAs promote tumor development by inhibiting tumor suppressor genes and/or genes that control cell differentiation or cell death. Many miRNAs have been reported that are significantly overexpressed in different cancers but only a few of them have been well characterized.

The miR-17 cluster This cluster is located at 13q31 which is amplified in lung cancer and several lymphomas. Compared with normal tissues, the expression of the miR-17 cluster is significantly increased in these types of cancers (41, 42). Overexpression of the miR-17 cluster using transgenic mice significantly accelerated the formation of lymphoid malignancies (42). Recent studies also indicated that the expression of the miR-17 cluster is related to the expression of the well-characterized oncogene, c-MYC. Their work shows that there is a negative feedback loop involving c-Myc, E2F1, miR-17-5p and miR-20a whereby c-Myc induces expression of E2F1 and the post-transcriptional repressors of E2F1; miR-17-5p and miR-20a (6, 43, 44).

miR-155 miR-155 is encoded within a region known as BIC, B-cell integration cluster, identified as a transcript derived from a common retroviral integration site for avian leucosis virus (45). B cells require miR-155 for normal production of isotype-switched, high-affinity antibodies and for memory response by targeting transcriptional regulator Pu.1 (46). miR-155 is up-regulated in different cancers such as certain B cell lymphomas (47), lung (48) and breast cancer (49). A study has recently shown in a transgenic mouse model that selective overexpression of miR-155 in B cells induces a polyclonal B-cell malignancy. In addition, a recent study indicated that the TP53INP1 gene, with anti-tumor activity, is a target of miR-155 (50). These studies strongly implicate miR-155 as an oncogene.

miR-372 and miR-373 Using a novel retroviral miRNA expression library, it was shown that overexpression of miR-372 and 373 can substitute for p53 loss and allow continued proliferation in the context of Ras activation (51). Furthermore, the study indicated these miRNAs neutralize p53-mediated CDK inhibition, possibly through direct inhibition of the expression of the tumor-suppressor LATS2. This suggests that these miRNAs are potential novel oncogenes participating in the development of human cancer by hampering the p53 pathway, thus allowing tumorigenic growth in the presence of wild-type p53.

miR-21 miR-21 was first discovered as a potential oncogene in glioblastoma because it was overexpressed in tumors and cancer cell lines (52). In addition, overexpression of miR-21 also is observed in various cancers including breast, colon, lung, pancreas, stomach and prostate (53). Knockdown of miR-21 in glioblastoma cell lines led to activation of caspases and a corresponding induction of apoptotic cell death (52). This result indicated that overexpression of miR-21 may promote tumorigenesis by inhibiting apoptosis. In addition, studies also have shown that miR-21 may target the programmed cell death 4 (PDCD4) and tumor suppressor gene tropomyosin 1 (TPM1) (54-56).

### Identification of MicroRNA Targets

Evidence for the involvement of miRNAs in cancer is very clear. The current challenge is to accurately identify the biological targets and therefore the functional effects of a miRNA. The effect that miRNAs exert on their targets results in repression of mRNA translation or enhanced mRNA degradation, although the opposite can occur under serum starvation (3, 57). This indicates that confirmation of the target genes of a miRNA will require both a transcriptomics and a proteomics approach.

At present, identification of targets for most miRNAs has been dependent on computational predictions, but these approaches are challenging due to the lack of strict base pairing between a miRNA and its target mRNA sequence. There are several microRNA target prediction algorithms available but the accuracy is quite low (less than 30%) (58). The basic principles of these predictions rely on several factors: complementarity to the 3′UTR of the target mRNA, strong binding of the 5′ end of the miRNA to the target, thermodynamic stability of the base pairing, conservation of the target mRNA 3′UTR miRNA binding sites, and lack of a strong secondary structure of the mRNA at the binding site of the miRNA. Experimental validation of miRNA targets is challenging because of the low accuracy of predictions of miRNA targets by computational prediction algorithms. So far, there is no simple and high-throughput assay for biologically validating miRNA targets. Currently the most common method involves cloning binding sites of the 3′ UTR of an endogenous mRNA fragment (or repeated fragments) into the 3′ region of a luciferase reporter plasmid and measuring whether expression of the construct in cells co-transfected with candidate miRNA is repressed. (10, 59, 60). A loss of function method has also been used in which a miRNA is inhibited by 2′-O-methyl-modified oligonucleotides, and the inhibition of activity is assayed either by luciferase activity or by gene expression analysis at the protein level (61, 62).

MiRNA-mediated translational inhibition depends on the stable physical association between the miRNA, RISC, and the target mRNA. Several groups have recently taken advantage of this interaction in vivo to identify mRNA targets. Immunoprecipitating the key component of RISC, AGO2, and then interrogating the total pulled down RNA on an expression microarray reveals de novo targets of miRNAs (63, 64). This approach provides a way to identify functional miRNA targets based on their physical interaction in vivo. Although these assays can be used to identify targets of miRNAs, the development of high-throughput target validation techniques will be necessary to raise the specificity and sensitivity of miRNA target prediction algorithms in the future.

Potential Therapeutic Applications of miRNAs

The analysis of miRNA expression profiles in cancer has revealed that aberrant expression of miRNAs is frequent and many tumor suppressor miRNAs are down-regulated in cancer. These tumor suppressor miRNAs are potential therapeutic targets for anticancer therapy. It might be possible to manipulate miRNA expression to inhibit cancer progression just as RNAi is being used in some approaches to gene therapy. A few studies have shown the potential utility of miRNA-based therapies in cancer. These include the induction of apoptosis by the miR-34 family in colon cancer cell lines (13) and by miR-15a/16-1 in CLL (27), inhibition of growth of cancer cells by let-7 (22, 23, 65), reduced migration and invasion by miR-125 in breast cancer cells (66) and the use of anti-AMOs to obtain a pro-apoptotic response in glioblastoma and breast cancer cells (52). Currently there no reported studies using miRNAs for in vivo anti-cancer therapy. However, the development of approaches for in vivo delivery of siRNA and short heteroduplex RNA (shRNA) to silence single target genes has established technical approaches also useful for miRNA therapy (67). Anti-cancer approaches based on systemic delivery of siRNAJshRNA in preclinical models have made use of viral vectors, liposomes, and nanoparticles (68-70). Some of the difficulties with the delivery of antisense and siRNA into cells will be faced in miRNA-based therapies. Introducing a polymer that is linear and charged across the membrane of a cell is difficult. The clear advantage miRNA-based gene therapy will have over siRNAs, shRNAs, and antisense oligonucleotides is that multiple miRNAs can be co-transcribed and each miRNA has multiple targets, such as let-7 which down-regulates RAS, MYC, and HMGA2 oncogenes (22, 23).

As mentioned above, re-expression of tumor suppressor miRNAs can inhibit cancer cell growth or promote cancer cell differentiation, both of which have therapeutic value. Synergistic activity of multiple miRNAs on the same mRNA has been demonstrated and has been indicated for endogenous targets (71, 72). The newly developed method to express multiple miRNAs from a single transcript to synergistically inhibit cancer cells by targeting multiple pathways involved in tumorigenesis is achieved as follows: 1) creation of a multiple miRNA expression vector able to target multiple oncogenic pathways by down-regulating many crucial genes involved in the aggressive behavior of many different types of cancer; 2) confirmation of the synergistic effects of multiple miRNA expression vector in vivo using mouse models; 3) and development a high throughput assay to identify the target genes of tumor suppressor miRNAs. The completion of these steps allow for the creation of a new class of vector for gene therapy based on miRNAs, providing an exciting first step towards the clinical application of miRNA therapy in cancer patients. Development of a high throughput assay to identify target genes of miRNAs, enables the gathering of important information about the exact biological effects of potential therapy in addition to providing an invaluable tool to the miRNA field. Finally, by using a combination of tumor suppressor miRNAs to target multiple pathways involved in tumorigenesis the miRNA vector has the potential to be a universal cancer therapy.

### SUMMARY OF THE INVENTION

In one embodiment, the invention relates to expression vectors comprising multiple miRNA families and clusters capable of targeting multiple oncogenic pathways by down-regulating many crucial genes involved in the aggressive behavior of many different types of cancer.

In another embodiment, the invention relates to methods of determining synergistic effects of multiple miRNA expression vectors in vivo.

In a related embodiment, the invention relates to methods of identifying target genes of tumor suppressor miRNAs using high throughput assays.

### BRIEF DESCRIPTION OF THE FIGURES

FIG. 1. FIG. 1. HCT116 colon cancer cells were transfected with pcDNA3.1(+) miRNA expression vectors containing either the individual miRNAs miR34a-V, miR34b-V, or miR34c-V, all three miRNAs together (miR34abc-V), or the empty vector (E.V.). (A) qPCR (real-time PCR) was conducted 48 hours post-transfection. Each reaction was done in duplicate. (B) Cell proliferation assays were conducted by transferring equal cell numbers to 10 cm dishes 48 hours post-transfection. After 13-14 days under G418 selection total cells were counted and normalized to the empty vector. (C) Colony formation assays were conducted by transferring equal cell numbers to 6-well plates 48 hours post-transfection.

FIG. 2. T24 bladder cancer cells were transfected with pcDNA3.1(+) miRNA expression vectors containing either miR-127 alone (miR127-V), the miR-127 cluster-V (miR-431, miR-433, miR-127, miR-432, and miR-136 in a single transcript), or the empty vector (E.V.). (A) Cell proliferation assays were conducted by transferring equal cell numbers to 10 cm dishes 48 hours post-transfection. After 13-14 days under G418 selection total cells were counted and normalized to the empty vector. (B) Colony formation assays were conducted by transferring equal number cells to 6-well plates 48 hours post-transfection. Colonies were stained and counted after 13-14 days under G418 selection and normalized to empty ,vector control.

### DETAILED DESCRIPTION OF THE INVENTION

The discovery of microRNAs (miRNAs), which are key regulators of gene expression involved in diverse cellular processes, was a breakthrough in the field of molecular biology. Aberrant expression of microRNAs (miRNAs), small ˜22 nucleotide non-coding RNAs, is involved in the initiation and progression of human cancer. miRNAs can act as either tumor suppressors or oncogenes by disrupting the expression of their target oncogenes or tumor suppressor genes, respectively. Molecular miRNA profiling has identified several miRNAs that act as either tumor suppressors by down-regulating oncogenes or as oncogenes by down-regulating tumor suppressor genes. The knockdown of an oncogene is a common strategy for gene therapy in cancer but most approaches target only one gene or one pathway, Unlike siRNA (short interfering RNA), each miRNA targets multiple genes. Therefore, a vector containing multiple tumor suppressor miRNAs are able to knockdown multiple target genes and pathways from a single transcript and could suppress tumorigenesis in an additive or synergistic manner. A flexible RNA polymerase II promoter-driven vector which expressed a single transcript containing three miRNA members of the miR-34 family has been developed. This multiple miRNA expression vector suppressed cancer cells in a synergistic manner compared to expression vectors with each miRNA individually. Likewise, the construction of an expression vector that contains multiple miRNAs not just from one family but containing multiple families or clusters of miRNAs (10 to 12 miRNAs total) that target different pathways involved in tumorigenesis has been developed.

The present invention allows for the creation of a new class of vector for gene therapy based on miRNAs, providing the first steps towards the clinical application of miRNA therapy in cancer patients. The development of a high throughput assay allows for the identification of target genes of miRNAs and for gathering of important information about the exact biological effects of potential therapy in addition to providing an invaluable tool to the miRNA field. By using a combination of tumor suppressor miRNAs to target multiple pathways involved in tumorigenesis the miRNA vector has the potential to be a universal cancer therapy.

Many microRNAs (miRNAs) have had their functional roles during tumorigenesis confirmed by in vitro and/or in vivo studies and are therefore considered to be strong candidate tumor suppressors and oncogenes. The invention allows for the development of novel classes of vectors for gene therapy based on miRNAs that are able to target multiple oncogenes and/or tumorigenic pathways in cancer. Additionally, the inclusion of a combination of miRNA families and clusters allows for expression vectors that are not specific to any cancer type but instead could be a universal cancer therapy. Using this approach, the inventors provide exciting steps towards the clinical application of miRNA therapy in cancer patients.

The development a multiple miRNA expression vector with synergistic inhibitory effects on cancer cells compared to individual miRNAs. The key step for the miRNA processing machinery to produce mature miRNAs seems to be the recognition of the hairpin structure and not the sequence outside of the pre-miRNA (73), implying that the sequence requirement for mature miRNA expression from an expression vector could be as little as a few base pairs in either direction of the pre-miRNA. Due to the small size of the pre-miRNA genes, it is technically simple to clone many pre-miRNA genes into the same expression vector. Therefore, it is possible to clone multiple tumor suppressor miRNAs into one vector able to affect many different pathways involved in tumorigenesis, creating a powerful miRNA-based universal cancer therapy. The inventors cloned the miR-34 tumor suppressor family (miR-34a, miR-34b and miR-34c), which is regulated by p53, into a single expression vector in order to determine whether it had a stronger inhibitory effect on cancer cell lines in comparison to the individual miRNAs. MiR-34a is located at chromosome 1p36, while miR-34b and miR-34c are located at chromosome 11q23, about 500 bp apart. Previous studies have shown that restored expression of individual miRNAs from the miR-34 family can induce apoptosis in cancer cell lines and inhibit cell growth (12). Because miR-34a, miR-34b, and miR-34c have similar roles when they are activated by p53, our strategy is to establish a synergistic expression vector by expressing 3 miRNAs (miR-34a, miR-34b, and miR-34c) from one single transcript. To create a multiple miRNA expression vector, approximately 50 by surrounding the pre-miRNAs for miR-34a, miR-34b, and miR-34c were amplified by PCR and then cloned into pcDNA3.1(+) either individually or all three together in one transcript of approximately 450 bp.

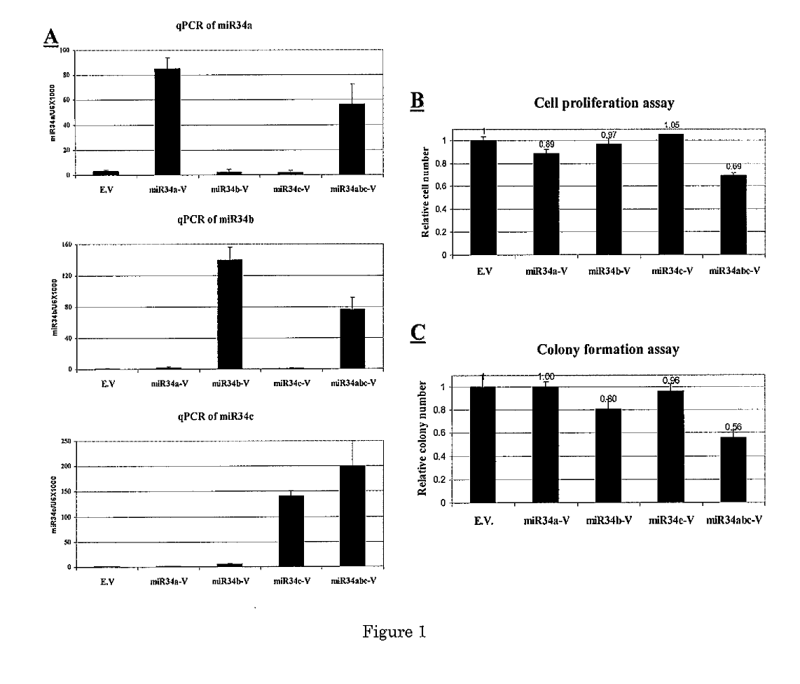

When HCT116 colon cancer cells, which have low levels of miR-34a, miR-34b, and miR-34c (12), were transfected, the miR-34abc vector yielded mature miRNAs at a level similar to each individual miRNA vector (FIG. 1A) as measured by stem-loop real-time PCR. Of the individual miR-34 family members, only miR-34a and miR-34b inhibited cell proliferation and colony formation, respectively (FIGS. 1B and C). However, the miR-34abc vector strongly inhibited both cell proliferation and colony formation, indicating that although each miR-34 might not have a strong effect individually when expressed together they have a powerful synergistic effect (FIGS. 1B and C).

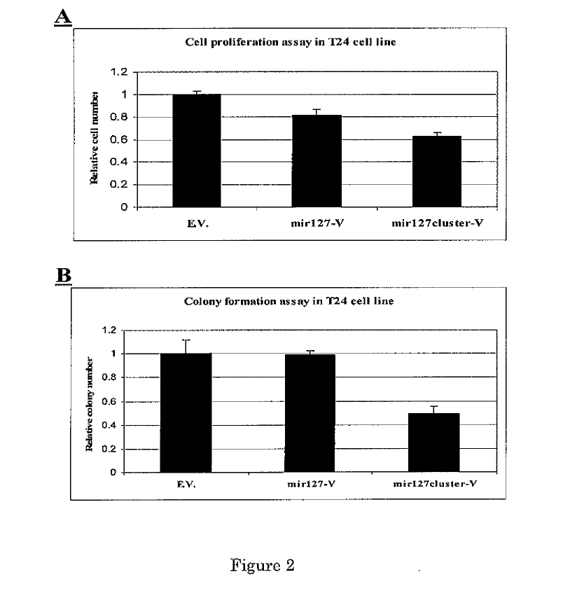

In addition, the inventors constructed an expression vector containing the miR-127 cluster, which consists of miR-431, miR-433, miR-127, miR-432, and miR-136 within a 4 kb genomic region. The inventors have previously shown that this cluster of miRNAs is expressed in normal tissues but not in bladder, colon or prostate cancers (10). One of these, miR-127, is embedded in a CpG island and was highly induced from its own promoter after treatment with the DNA methylation inhibitor and chromatin-modifying drugs 5-Aza-CdR and PBA, respectively. The invetors study also indicated that miR-127 can down-regulate the pro-oncogene BCL6, making it a potential tumor suppressor miRNA (10). Since the miR-127 cluster, not miR-127 alone, is silenced in cancer, we established an expression vector with an insert of ˜800 bp containing the 5 miRNAs in a single transcript to compare its efficacy to miR-127 alone in the bladder cancer cell line T24, which does not express the miR-127 cluster. Once again, the vector expressing the miR-127 cluster strongly inhibited both cell proliferation and colony formation when compared with miR-127 alone (FIGS. 2A and B).

Taken together, these results confirm that expression of multiple miRNAs is more effective at inhibiting cancer cell lines than individual miRNAs. The next step is to determine whether expression of multiple families or clusters of miRNAs have stronger inhibitory effects in cancer cells than single miRNA families or clusters. The inventors believe that these findings represent a new way to treat cancer. In order to understand more fully the biological impact this multiple miRNA expression vector have as a cancer therapy, a high-throughput method to identify additional mRNA targets of the included tumor suppressor miRNAs is used.

The development of approaches for in vivo delivery of short interfering RNA (siRNA) to silence a single target gene has established techniques that are also useful for miRNA delivery. The inventors have focused on the ability of a single miRNA to down-regulate many crucial genes or pathways involved in the aggressive behavior of cancer. By linking many miRNAs together into a single vector, the inventors are able to suppress vast numbers of target genes at once. Two multiple miRNA expression vectors containing the miR-34abc or the miR-127 cluster, both of which had a synergistic inhibitory effect on cancer cell lines compared to expression vectors containing individual miRNAs have been successfully made (FIGS. 1 and 2). An expression vector containing between 10 to 12 miRNAs from multiple miRNA families and clusters allows for more robust anti-cancer effects in cancer cell lines and in a mouse model has been created. Furthermore, the development of a high-throughput target validation assay allows for the identification of miRNA target genes using the multiple miRNA expression vectors.

Development of a miRNA expression vector containing multiple miRNA families and clusters that target different oncogenic pathways and confirm the synergistic effects of the multiple microRNA expression vector over single miRNA vectors in various human cancer cell lines.

Preliminary studies, show successful synergistic effects of multiple miRNA expression vectors are made by ligating individual miRNAs of a tumor suppressor microRNA family or cluster into one expression vector. The inventors have created expression vectors containing multiple miRNA families and clusters. Then synergistic inhibitory effects of the vectors in various human cancer cell lines including bladder cancer (T24, UMUC3, RT4), prostate cancer (PC3, LNCaP, DU145), colon cancer (HCT116, LoVo, RKO), breast cancer (MCF7,MDA-MB-453, MDA-MB-361), lung cancer (A549, H1299), and leukemia (K562, Jurkat, U937) are tested. Normal cell lines such as LD419 are included in this experiment as controls for the unintended effects of miRNAs. Studies have indicated that miRNA expression profiles vary by tissue and by cancer type (74, 1). Therefore, different cancer cell lines have different responses to a single miRNA or even to a single miRNA cluster or family. The final goal is to combine multiple tumor suppressor miRNAs found to be involved in many different types of cancer into one expression vector that has robust anti-tumor effects on most, if not all, cancers.

### Materials and Methods

Cell lines. Bladder cancer (T24, UMUC3, RT4), prostate cancer (PC3, LNCaP, DU145), colon cancer (HOT116, LoVo, RKO), breast cancer (MCF7,MDA-MB-453, MDA-MB-361), lung cancer (A549, H1299), and leukemia (K562, Jurkat, U937) cell lines will be used in this study. Some of the cell lines such as T24, UMUC, RT4, and MCF7, PC3 are available in the lab; the others are obtained from American Type Culture Collection (Rockville, Md.). Culture conditions will follow the instructions of ATCC.

Create expression vectors with multiple miRNA tumor suppressors. Expression vectors are made by PCR amplifying 50 to 100 by surrounding the pre-miRNAs (10 to 12) and cloning these separately into multiple restriction sites of pcDNA3.1(+) (Invitrogen) resulting in an insert of less than 2 kb containing 10 to 12 miRNAs. The inventors only include let-7b and let-7e as members of the let-7 family because they are the most divergent (77) of the 16 family members.

Cellular proliferation. The comparison of colony and cell counts between empty vector control and miRNA expression vectors are done using Dunnet's Method (78). Briefly, the analysis is based on log-transformed data where means and 95% confidence intervals are calculated and transformed back to the original scale. Cell doubling time and a focus-forming assay is performed to measure cell growth in the cells with or without multiple miRNA expression vectors to identify tumor supressor properties in vivo (79, 80). The cell proliferation assays are conducted in triplicate as described previously (81). Each well is trypsinized and equal cell numbers plated onto 10 cm dishes with medium containing G418 (Sigma). Medium is changed every 3-4 days and total cell numbers counted after 13-14 days.

Colony formation assays are conducted as described previously (82). 48 hours after transfection equal numbers of cells are plated in triplicate into 6-well dishes containing medium with G418 (Sigma) at the same concentrations as the cell proliferation assay. Medium is changed every 3-4 days and colonies counted after 13-14 days by washing with PBS, fixing with methanol and staining with Giemsa.

DNA fragmentation and apoptosis assay. As mentioned before, some of miRNAs including in the expression vector can induce apoptosis. Apoptosis is measured in various cancer cell lines with or without multiple miRNAs expression vector using the In Site Cell Death Detection Kit (TUNEL assay) from Roche.

Invasion assay. Cellular potential for invasiveness is determined using six-well Matrigel invasion chambers (BD Biosciences Discovery

Labware). Cells are seeded into upper inserts at 2×105 per insert in serum-free DMEM and outer wells are filled with DMEM containing 5% FBS as chemoattractant. Cells are incubated at 37° C. with 5% carbon dioxide for 48 h, and then noninvading cells are removed by swabbing the top layer of the Matrigel with a Q-tip. The membrane containing invading cells is stained with hematoxylin for 3 min, washed, and mounted on slides. The entire membrane with invading cells are counted under a light microscope at 40× objective.

Western blots. Cells are harvested by treatment with trypsin and resuspended in WA buffer. The resuspended cells are lysed by 2 cycles of sonication for 15 sec. Equal amounts of protein (20-50 μg) are separated on SDS-polyacrylamide gels and transferred to PVDF membranes. The blot is probed with antibodies against the potential target protein and control protein and Image of individual proteins are visualized using ECL detection system (Amersham Biosciences, Piscataway, N.J.) (80).

Reverse transcription and Taqman real-time PCR. RNA is isolated from cell lines using Trizol (Invitrogen, Carlsbad, Calif.) according to the manufacturer's protocol. All reagents for miRNA Taqman assays to detect mature miRNAs are purchased from Applied Biosystems (Foster City, Calif.) and used according to the manufacturer's protocol (83). U6 is used as the internal control and all reactions are done in duplicate.

Confirmation of the synergistic effect of a multiple microRNA expression vector over single microRNA vectors on cancer in vivo using mouse models.

Based on the results from above, 4 to 6 different cancer cell lines that are able to form xenograft tumors into nude mice after transfection with the multiple miRNA expression vector to test the effects in vivo are injected into mice. The animal experiments used are standardized.

Animal experiments. Animal studies are performed according to institutional guidelines. Cancer cell lines of different tissue types (4-6 cell lines) are transfected in vitro with 100 nM (final concentration) of the control expression vector or the multiple miRNA expression vector DNA by using Lipofectamine 2000 reagent (Invitrogen), according to the protocol of the manufacturer. At 48 after transfection, 0.5 to 3×106 cells (injection) are inoculated subcutaneously into the right and left flanks (along the midaxillary lines) of 4- to 6-week-old male BALB/c nu/nu mice (Harlan, San Diego, Calif.). In order to obtain statistically meaningful results, at least six mice per group (control and 6 cancer cell lines) are used. Tumor diameters are measured 7 days after injection and every 5 days thereafter. After 3 weeks (the days might be various based on the cell lines), mice are killed and tumors are weighted after necropsy. Tumor volumes are determined using the equation V (in mm3)=A×B2/2, where A is the largest diameter and B is the perpendicular diameter. Tumors are removed and each tumor is divided into two separate portions. One portion is immediately fixed with neutral buffered formalin, embedded in OCT compound, frozen, and then sectioned. The frozen sections are stained with hematoxylin and eosin. All histologic examinations are carried out by light microscopy using a Leica DM LB microscope (Leica Microsystems, Inc., Bannockburn, Ill.). The other potion of each tumor is used for isolating DNA and total RNA for analysis of DNA methylation by Ms-SNuPE, which was developed in the inventors lab (84), and of miRNAs and related gene expression by stem loop RT-PCR or real-time RT-PCR, respectively.

Identification of target genes of the tumor suppressor microRNAs from our multiple microRNA expression vector by transfecting cells, screening for down-regulated mENAs by microarray, and enriching target mRNAs using RISC immunoprecipitation (RIP) and identifying the mRNAs by microarray (RIP on chip). Confirmation of potential target genes from microarray results by prediction algorithms, Western blot, real-time RT-PCR, and luciferase assay.

Although the inventors expect the multiple miRNA expression vector to inhibit tumor cell growth, knowing the exact gene targets of the tumor suppressor miRNAs helps to understand the mechanism behind any synergistic effects. Furthermore, since the final goal is to use this expression vector for treatment for human cancers, identifying potential target genes helps to predict the consequences of this therapy such as any potential side-effects due to up-regulating harmful genes or down-regulating beneficial genes in normal cells. Experimental validation of miRNA targets is challenging because of the low accuracy (˜30%) of miRNA target prediction algorithms (58). There is a need for a simple and high-throughput assay for biologically validating miRNA targets. The miRNA:mRNA association is mediated by the RISC complex, the most important member of which is AGO2. The inventors are able to identify de novo miRNA:mRNA interactions by immunoprecipitating AGO2 and isolate the accompanying RNA (63, 85). As described above, the inventors interrogate the enriched mRNA with an expression array in order to determine potential target genes and screen out background levels using mRNA from cells transfected with the empty control vector. Potential targets are confirmed by real time RT-PCR, Western blots, microRNA target prediction algorithms, and/or luciferase assay. This approach allows for the establishment of a novel high-throughput assay for validating miRNA targets and be especially useful in identifying the exact targets of the tumor suppressor miRNAs in the expression vector.

Coimmunoprecipitation of AGO2 and mRNA Targets. This assay takes advantage of the RISC-miRNA-mRNA interaction necessary for gene repression and coimmunoprecipitates AGO-2, a component of the RISC complex, and target mRNAs containing miRNA binding sites (64). Cells with either the multiple miRNA expression vector or a control vector and prepare extracts are transfected. Cells are harvested 48 h after transfection and washed in PBS followed by hypotonic lysis buffer [10 mM Tris, pH 7.5, 10 mM KCl, 2 mM MgCl2, 5 mM DTT, and 1 tablet per 10 ml of protease inhibitors, EDTA-free (Roche)]. Cells are incubated in lysis buffer for 15 min and lysed by douncing. Immediately after doucing, the lysates are supplemented with 5×ATP depletion mix [4 units/μl RNaseIn (Promega), 100 mM glucose, 0.5 unites/μl hexokinase (Sigma), 1 mg/ml yeast tRNA (Invitrogen), 450 mM KCl] to a final concentration of 1×. The lysates are cleared by centrifugation at 16,000×g for 30 min at 40C. Before immunoprecipitation, anti AGO2 (elF2C) (sc-32877, Santa Cruz Biotechnology, Inc) is pre-blocked for 30 min in wash buffer [0.5% Nonidet P-40, 150 mM NaCl, 2 mM MgCl2, 2 mM CaCl2, 20 mM Tris, pH 7.5, 5 mM DTT, and 1 tablet per 10 ml of protease inhibitors] supplemented with 1 mg/ml yeast tRNA and 1 mg/ml BSA, followed by a wash in wash buffer. One volume of wash buffer is added to the lysates, and AGO2 is immunoprecipitated with pre-blocked beads for 4 h at 4° C. The beads are washed once with wash buffer and twice in wash buffer containing 650 mM NaCl, the slurry is transferred to a new tube on the last wash, and bound RNA is extracted with TRIzol.

Mieroarray analysis. Total RNA or RNA from AGO2 coimmunoprecipiation is isolated from cells transfected with either the multiple miRNA expression vector or a control vector using TRIzol. To look at global gene expression RNA is hybridrized to the human 6 v2 Expression BeadChip (Illumina) and data analysis is performed using Illumina software by the Epigenome Center on a fee-for service-basis.

MicroRNA target prediction algorithms. The potential target genes are first confirmed by the following four prediction algorithms:
Mirnaviewer (http://cbio.mskcc.org/mirnaviewer/);
PicTar(http://pictar.bio.nyu.edu/);
TargetScan4.1(http://www.targetscan.org;and
PITA(http://genie.weizmann.ac.il/pubs/mir07/mir07_data.html). This analysis is performed by the Epigenome Center on a fee-for service-basis.

Western blots. The same as above.

Real-time RT-PCR. Targets are be confirmed by real-time RT-PCR. RNA is reverse-transcribed using 2 μg of RNA and random hexamers, deoxy nucleotide triphosphates (Boehringer Mannheim, Germany) and Superscript II reverse transcriptase (Life Technologies, Inc., Palo Alto, Calif.) in a 50 μl reaction. The mixture is placed at room temperature for 10 min, 42° C. for 45 min, and 90° C. for 3 min, then rapidly cooled to 0° C. The resulting cDNA is amplified with primers specific to the gene of interest with β-actin or GAPDH as a control. Quantitative PCR is performed on the DNA Engine Opticon System (MJ Research, Cambridge, Mass.) using AmpliTaq Gold DNA polymerase (Applied Biosystems) with 2μl cDNA, gene specific primers, and fluorescently labeled TaqMan probes synthesized by BioResarch. All PCRs is carried out under the same conditions: 95° C. for 15 s and 59° C. for 1 min for 45 cycles (86).

Luciferase assay. The luciferase assay is performed in order to further confirm the identity of miRNA target genes and determine the miRNA binding site in the target gene. This assay has been used in the inventors' lab (10). Briefly, luciferase constructs are made by ligating oligonucleotides containing the wild type or mutant target site of the identified gene's 3′UTR into the XbaI site of pGL3-control vector (Promega). Cells both with and without expression of the miRNA is transfected with 0.4 μg of firefly luciferase reporter vector containing a wild-type or mutant target site and 0.02 μg of the control vector containing Renilla luciferase, pRL-CMV (Promega), using Lipofectamine 2000 (Invitrogen). Luciferase assays are performed 48 h after transfection using the Dual Luciferase Reporter Assay System (Promega). Firefly luciferase activity is normalized to Renilla luciferase.

Many modifications and variation of the invention as hereinbefore set forth can be made without departing from the spirit and scope thereof and therefore only such limitations should be imposed as are indicated by the appended claims.

All patent and literature references cited in the present specification are hereby incorporated by reference in their entirety.

### REFERENCES

1. Galin G A, Croce C M. MicroRNA signatures in human cancers. Nat Rev Cancer 2006;6(11):857-66.

2. Valencia-Sanchez M A, Liu J, Hannon G J, Parker R. Control of translation and mRNA degradation by miRNAs and siRNAs. Genes Dev 2006; 20 (5):515-24.

3. Vasudevan S, Tong Y, Steitz J A. Switching from Repression to Activation: MicroRNAs Can Up-Regulate Translation. Science 2007.

4. Lim L P, Lau N C, Garrett-Engele P, Grimson A, Schelter J M, Castle J, et al. Microarray analysis shows that some microRNAs downregulate large numbers of target mRNAs. Nature 2005;433(7027):769-73.

5. Ambros V. The functions of animal microRNAs. Nature 2004; 431(7006):350-5.

6. O'Donnell K A, Wentzel E A, Zeller K I, Dang C V, Mendell J T. c-Myc-regulated microRNAs modulate E2F1 expression. Nature 2005;435(7043): 839-43.

7. Calin G A, Sevignani C, Dumitru C D, Hyslop T, Noch E, Yendamuri S, et al. Human microRNA genes are frequently located at fragile sites and genomic regions involved in cancers. Proc Natl Acad Sci USA 2004;101(9):2999-3004.

8. Calin G A, Croce C M. MicroRNAs and chromosomal abnormalities in cancer cells. Oncogene 2006;25(46):6202-10.

9. Zhang L, Huang J, Yang N, Greshock J, Megraw M S, Giannakakis A, et al., microRNAs exhibit high frequency genomic alterations in human cancer. Proc Natl Acad Sci USA 2006;103(24):9136-41.

10. Saito Y, Liang G, Egger G, Friedman J M, Chuang J C, Coetzee G A, et al. Specific activation of microRNA-127 with downregulation of the proto-oncogene BCL6 by chromatin-modifying drugs in human cancer cells. Cancer Cell 2006;9(6):435-43.

11. He L, He X, Lim L P, de Stanchina E, Xuan Z, Liang Y, et al. A microRNA component of the p53 tumour suppressor network. Nature 2007;447(7148):1130-4.

12. Raver-Shapira N, Marciano E, Meiri E, Spector Y, Rosenfeld N, Moskovits N, et al. Transcriptional activation of miR-34a contributes to p53-mediated apoptosis. Mol Cell 2007;26(5):731-43.

13. Chang T C, Wentzel E A, Kent O A, Ramachandran K, Mullendore M, Lee KH, et al. Transactivation of miR-34a by p53 broadly influences gene expression and promotes apoptosis. Mol Cell 2007;26(5):745-52.

14. Wei C L, Wu Q, Vega V B, Chiu K P, Ng P, Zhang T, et al. A global map of p53 transcription-factor binding sites in the human genome. Cell 2006;124(1):207-19.

15. He L, He X, Lowe S W, Hannon G J. microRNAs join the p53 network—another piece in the tumour-suppression puzzle. Nat Rev Cancer 2007;7(11):819-22.

16. Versteeg R, Caron H, Cheng N C, van der Drift P, Slater R, Westerveld A, et al. 1p36: every subband a suppressor? Eur J Cancer 1995; 31A(4):538-41.

17. Welch C, Chen Y, Stallings R L. MicroRNA-34a functions as a potential tumor suppressor by inducing apoptosis in neuroblastoma cells. Oncogene 2007;26(34):5017-22.

18. Gaur A, Jewell D A, Liang Y, Ridzon D, Moore J H, Chen C, et al. Characterization of microRNA expression levels and their biological correlates in human cancer cell lines. Cancer Res 2007;67(6):2456-68.

19. Soussi T. p53 alterations in human cancer: more questions than answers. Oncogene 2007;26(15):2145-56.

20. Pasquinelli A E, Reinhart B J, Slack F, Martindale M Q, Kuroda M I, Mailer B, et al. Conservation of the sequence and temporal expression of let-7 heterochronic regulatory RNA. Nature 2000;408(6808):86-9.

21. Takamizawa J, Konishi H, Yanagisawa K, Tomida S, Osada H, Endoh H, et al. Reduced expression of the let-7 microRNAs in human lung cancers in association with shortened postoperative survival. Cancer Res 2004;64(11):3753-6.

22. Johnson S M, Grosshans H, Shingara J, Byrom M, Jarvis R, Cheng A, et al. RAS is regulated by the let-7 microRNA family. Cell 2005;120(5):635-47.

23. Mayr C, Hemann M T, Bartel D P. Disrupting the pairing between let-7 and Hmga2 enhances oncogenic transformation. Science 2007;315(5818):1576-9.

24. Yu F, Yao H, Zhu P, Zhang X, Pan Q, Gong C, et al. let-7 Regulates Self Renewal and Tumorigenicity of Breast Cancer Cells. Cell 2007;131(6):1109-23.

25. Calin G A, Dumitru C D, Shimizu M, Bichi R, Zupo S, Noch E, et al. Frequent deletions and down-regulation of micro-RNA genes miR15 and miR16 at 13q14 in chronic lymphocytic leukemia. Proc Nati Acad Sci USA 2002;99(24):15524-9.

26. Dong J T, Boyd J C, Frierson H F, Jr. Loss of heterozygosity at 13q14 and 13q21 in high grade, high stage prostate cancer. Prostate 2001;49(3):166-71.

27. Cimmino A, Calin G A, Fabbri M, Iorio M V, Ferracin M, Shimizu M, et al. miR-15 and miR-16 induce apoptosis by targeting BCL2. Proc Nati Acad Sci USA 2005;102(39):13944-9.

28. Akao Y, Nakagawa Y, Naoe T. MicroRNA-143 and -145 in colon cancer. DNA Cell Biol 2007;26(5):311-20.

29. Akao Y, Nakagawa Y, Kitade Y, Kinoshita T, Naoe T. Downregulation of microRNAs-143 and -145 in B-cell malignancies. Cancer Sci 2007;98(12):1914-20.

30. Shi B, Sepp-Lorenzino L, Prisco M, Linsley P, deAngelis T, Baserga R. Micro RNA 145 targets the insulin receptor substrate-1 and inhibits the growth of colon cancer cells. J Biol Chem 2007;282(45):32582-90.

31. Jones P A, Baylin S B. The fundamental role of epigenetic events in cancer. Nat Rev Genet 2002;3(6):415-28.

32. Jones P A, Baylin S B. The epigenomics of cancer. Cell 2007; 128(4):683-92.

33. Egger G, Liang G, Aparicio A, Jones P A. Epigenetics in human disease and prospects for epigenetic therapy. Nature 2004;429(6990):457-63.

34. Fabbri M, Garzon R, Cimmino A, Liu Z, Zanesi N, Callegari E, et al. MicroRNA-29 family reverts aberrant methylation in lung cancer by targeting DNA methyltransferases 3A and 3B. Proc Natl. Acad Sci USA 2007; 104(40): 15805-10.

35. Kim V N. MicroRNA biogenesis; coordinated cropping and dicing. Nat Rev Mol Cell Biol 2005;6(5):376-85.

36. Mi 5, Lu J, Sun M, Li Z, Zhang H, Neilly M B, et al. MicroRNA expression signatures accurately discriminate acute lymphoblastic leukemia from acute myeloid leukemia. Proc Nati Acad Sci USA 2007; 104(50): 19971-6.

37. Fazi F, Racanicchi S, Zardo G, Starnes L M, Mancini M, Travaglini L, et al. Epigenetic silencing of the myelopoiesis regulator microRNA-223 by the AML1IETO oncoprotein. Cancer Cell 2007;12(5):457-66.

38. Lu L, Katsaros D, de la Longrais I A, Sochirca O, Yu H. Hypermethylation of let-7a-3 in epithelial ovarian cancer is associated with low insulin-like growth factor-II expression and favorable prognosis. Cancer Res 2007;67(21):10117-22.

39. Meng F, Wehbe-Janek H, Henson R, Smith H, Patel T. Epigenetic regulation of microRNA-370 by interleukin-6 in malignant human cholangiocytes. Oncogene 2007.

40. Lujambio A, Ropero S, Ballestar E, Fraga M F, Cerrato C, Setien F, et al. Genetic unmasking of an epigenetically silenced microRNA in human cancer cells. Cancer Res 2007;67(4):1424-9.

41. Hayashita Y, Osada H, Tatematsu Y, Yamada H, Yanagisawa K, Tomida S, et al. A polycistronic microRNA cluster, miR-17-92, is overexpressed in human lung cancers and enhances cell proliferation. Cancer Res 2005;65(21):9628-32.

42. He L, Thomson J M, Hemann M T, Hernando-Monge E, Mu D, Goodson S, et al. A microRNA polycistron as a potential human oncogene. Nature 2005;435(7043):828-33.

43. Hammond S M. MicroRNAs as oncogenes. Curr Opin Genet Dev 2006;16(1):4-9.

44. Woods K, Thomson J M, Hammond S M. Direct regulation of an oncogenic micro-RNA cluster by E2F transcription factors. J Biol Chem 2007;282(4):2130-4.

45. Tam W, Ben-Yehuda D, Hayward W S. bic, a novel gene activated by proviral insertions in avian leukosis virus-induced lymphomas, is likely to function through its noncoding RNA. Mol Cell Biol 1997; 17(3):1490-502.

46. Vigorito E, Perks K L, Abreu-Goodger C, Bunting S, Xiang Z, Kohlhaas S, et al. microRNA-155 Regulates the Generation of Immunoglobulin Class-Switched Plasma Cells. Immunity 2007;27(6):847-59.

47. Eis P S, Tam W, Sun L, Chadburn A, Li Z, Gomez M F, et al. Accumulation of miR-155 and BIC RNA in human B cell lymphomas. Proc Nati Acad Sci USA 2005;102(10):3627-32.

48. Yanaihara N, Caplen N, Bowman E, Seike M, Kumamoto K, Yi M, et al. Unique microRNA molecular profiles in lung cancer diagnosis and prognosis. Cancer Cell 2006;9(3):189-98.

49. Iorio M V, Ferracin M, Liu C G, Veronese A, Spizzo R, Sabbioni S, et al. MicroRNA gene expression deregulation in human breast cancer. Cancer Res 2005;65(16):7065-70.

50. Gironella M, Seux M, Xie M J, Cano C, Tomasini R, Gommeaux J, et al. Tumor protein 53-induced nuclear protein 1 expression is repressed by miR-155, and its restoration inhibits pancreatic tumor development. Proc Nati Aced Sci USA 2007;104(41);16170-5.

51. Voorhoeve P M, le Sage C, Schrier M, Gillis A J, Stoop H, Nagel R, et al. A genetic screen implicates miRNA-372 and miRNA-373 as oncogenes in testicular germ cell tumors. Cell 2006;124(6):1169-81.

52. Chan J A, Krichevsky A M, Kosik K S. MicroRNA-21 is an antiapoptotic factor in human glioblastoma cells. Cancer Res 2005;65(14): 6029-33.

53. Volinia S, Calin G A, Liu C G, Ambs S, Cimmino A, Petrocca F, et al. A microRNA expression signature of human solid tumors defines cancer gene targets. Proc Nati Acad Sci USA 2006;103(7):2257-61.

54. Frankel L B, Christoffersen N R, Jacobsen A, Lindow M, Krogh A, Lund All. Programmed cell death 4 (PDCD4) is an important functional target of the microRNA miR-21 in breast cancer cells. J Biol Chem 2007.

55. Asangani I A, Rasheed S A, Nikolova D A, Leupold J H, Colburn N H, Post S, et al. MicroRNA-21 (miR-21) post-transcriptionally downregulates tumor suppressor Pdcd4 and stimulates invasion, intravasation and metastasis in colorectal cancer. Oncogene 2007.

56. Zhu S, Si M L, Wu H, Mo Y Y. MicroRNA-21 targets the tumor suppressor gene tropomyosin 1 (TPM1). J Biol Chem 2007;282(19):14328-36.

57. Lai E C. Micro RNAs are complementary to 3′ UTR sequence motifs that mediate negative post-transcriptional regulation. Nat Genet 2002; 30(4):363-4.

58. Chaudhuri K, Chatterjee R. MicroRNA detection and target prediction: integration of computational and experimental approaches. DNA Cell Biol 2007;26(5):321-37.

59. Doench J G, Petersen C P, Sharp P A. siRNAs can function as miRNAs. Genes Dev 2003;17(4):438-42.

60. Pillai R S, Bhattacharyya S N, Artus CG, Zoller T, Cougot N, Basyuk E, et al. Inhibition of translational initiation by Let-7 MicroRNA in human cells. Science 2005;309(5740):1573-6.

61. Chen J F, Mandel E M, Thomson J M, Wu Q, Callis T E, Hammond S M, et al. The role of microRNA-1 and microRNA-133 in skeletal muscle proliferation and differentiation. Nat Genet 2006;38(2):228-33.

62. Schratt G M, Tuebing F, Nigh E A, Kane C G, Sabatini M E, Kiebler M, et al. A brain-specific microRNA regulates dendritic spine development. Nature 2006;439(7074):283-9.

63. Easow G, Teleman A A, Cohen S M. Isolation of microRNA targets by miRNP immunopurification. Rna 2007;13(8):1198-204.

64. Karginov F V, Conaco C, Xuan Z, Schmidt B H, Parker J S, Mandel G, et al. A biochemical approach to identifying microRNA targets. Proc Nati Acad Sci USA 2007;104(49):19291-6.

65. Akao Y, Nakagawa Y, Naoe T. let-7 microRNA functions as a potential growth suppressor in human colon cancer cells. Biol Pharm Bull 2006;29(5):903-6.

66. Scott G K, Goga A, Bhaumik D, Berger C E, Sullivan C S, Benz C C. Coordinate suppression of ERBB2 and ERBB3 by enforced expression of micro-RNA miR-125a or miR-125b. J Biol Chem 2007;282(2):1479-86.

67. Devi G R. siRNA-based approaches in cancer therapy. Cancer Gene Ther 2006;13(9):819-29.

68. Abbas-Terki T, Blanco-Bose W, Deglon N, Pralong W, Aebischer P. Lentiviral-mediated RNA interference. Hum Gene Ther 2002;13(18):2197-201.

69. Lu P Y, Xie F, Woodle M C. In vivo application of RNA interference: from functional genomics to therapeutics. Adv Genet 2005;54:117-42.

70. Tong A W. Small RNAs and non-small cell lung cancer. Curr Mol Med 2006;6(3):339-49.

71. Doench J G, Sharp P A. Specificity of microRNA target selection in translational repression. Genes Dev 2004;18(5):504-11.

72. Stark A, Brennecke J, Bushati N, Russell R B, Cohen S M. Animal MicroRNAs confer robustness to gene expression and have a significant impact on 3′UTR evolution. Cell 2005;123(6):1133-46.

73. Han J, Lee Y, Yeom K H, Nam J W, Heo I, Rhee J K, et al. Molecular basis for the recognition of primary microRNAs by the Drosha-DGCR8 complex. Cell 2006;125(5):887-901.

74. Bommer G T, Gerin I, Feng Y, Kaczorowski A J, Kuick R, Love R E, et at p53-mediated activation of miRNA34 candidate tumor-suppressor genes. Curr Biol 2007;17(15):1298-307.

75. Brown B D, Cantore A, Annoni A, Sergi L S, Lombardo A, Della Valle P, et al. A microRNA-regulated lentiviral vector mediates stable correction of hemophilia B mice. Blood 2007;110(13):4144-52.

76. Egger G, Jeong S, Escobar S G, Cortez C C, Li T W, Saito Y, et al. Identification of DNMT1 (DNA methyltransferase 1) hypomorphs in somatic knockouts suggests an essential role for DNMT1 in cell survival. Proc Nati Acad Sci USA 2006;103(38):14080-5.

77. Lee Y S, Dutta A. The tumor suppressor microRNA let-7 represses the HMGA2 oncogene. Genes Dev 2007;21(9):1025-30.

78. Dunnet C W. A multiple comparisons procedure for comparing several treatments with a control. Journal of the American Statistical Association 1955;50:1096-1121.

79. Cheng J C, Yoo C B, Weisenberger D J, Chuang J, Wozniak C, Liang G, et al. Preferential response of cancer cells to zebularine. Cancer Cell 2004;6(2):151-8.

80. Cheng J C, Weisenberger D J, Gonzales F A, Liang G, Xu G L, Hu Y G, et al. Continuous zebularine treatment effectively sustains demethylation in human bladder cancer cells. Mol Cell Biol 2004;24(3):1270-8.

81. Robertson K D, Jones P A. Tissue-specific alternative splicing in the human INK4a/ARF cell cycle regulatory locus. Oncogene 1999; 18(26):3810-20.

82. Kim T Y, Zhong S, Fields C R, Kim J H, Robertson K D. Epigenomic profiling reveals novel and frequent targets of aberrant DNA methylation-mediated silencing in malignant glioma. Cancer Res 2006;66(15): 7490-501.

83. Chen C, Ridzon D A, Broomer A J, Zhou Z, Lee DH, Nguyen J T, et al. Real-time quantification of microRNAs by stem-loop RT-PCR. Nucleic Acids Res 2005;33(20):e179.

84. Gonzalgo M L, Liang G. Methylation-sensitive single-nucleotide primer extension (Ms-SNuPE) for quantitative measurement of DNA methylation. Nat Protoc 2007;2(8):1931-6.

85. Schwarz D S, Zamore P D. Why do miRNAs live in the miRNP? Genes Dev 2002;16(9):1025-31.

86. Liang G, Lin J C, Wei V, Yoo C, Cheng J C, Nguyen C T, et al, Distinct localization of histone H3 acetylation and H3-K4 methylation to the transcription start sites in the human genome. Proc Nati Acad Sci USA 2004;101(19):7357-62.

What is claimed is:

1. An expression vector comprising multiple miRNA families and/or clusters, wherein said families and/or clusters are capable of inhibiting the effects of multiple cancers.

2. The expression vector according to claim 1, wherein said multiple miRNA family and/or cluster comprises 2 or more of the group of the miR-34 family, let-7 family, miR-15a and miR-16-1 cluster, miR-143 and miR-145 cluster, miR-29 family, miR-127 cluster, miR-17 cluster, miR-155 cluster, miR-372 cluster and miR-373 cluster, and miR-21 cluster.

3. The expression vector according to claim 1, wherein said cancer comprises 2 or more of the group of bladder, prostate, colon, breast, lung, and leukemia.

4. The expression vector according to claim 1, wherein said family and/or cluster down-regulates one or more cancer related genes.

5. An expression vector comprising multiple miRNA families and/or clusters, wherein said families and/or clusters are capable of synergistically inhibiting the effects of multiple cancers as compared to an individual miRNA.

6. The expression vector according to claim 5, wherein said synergistic inhibitory effects comprises 2 or more of the group of cell proliferation, colony formation, DNA fragmentation and apoptosis, and invasion.

7. The expression vector according to claim 5, wherein said multiple miRNA family and/or cluster comprises 2 or more of the group of the miR-34 family, let-7 family, miR-15a and miR-16-1 cluster, miR-143 and miR-145 cluster, miR-29 family, miR-127 cluster, miR-17 cluster, miR-155 cluster, miR-372 cluster and miR-373 cluster, and miR-21 cluster.

8. The expression vector according to claim 5, wherein said cancer comprises 2 or more of the group of bladder, prostate, colon, breast, lung, and leukemia.

9. The expression vector according to claim 5, wherein said family and/or cluster down-regulates one or more cancer related genes.
